(19) United States
(12) Patent Application Publication
(10) Pub. No.: US 2007/0021743 A1
(43) Pub. Date: Jan. 25, 2007

(54) Compressible/expandable hydrophilic ablation electrode

(75) Inventors: Robert F. Rioux, Ashland, MA (US); Paul DiCarlo, Middleboro, MA (US)

Correspondence Address: Vista IP Law Group LLP, 2040 MAIN STREET, 9TH FLOOR, IRVINE, CA, 92614, US

(73) Assignee: BOSTON SCIENTIFIC SCIMED, INC., Maple Grove, MN (US)

(21) Appl. No.: 11/187,246

(22) Filed: Jul. 22, 2005

(51) Int. Cl. A61B 18/14

(52) U.S. Cl. 606/32; 606/41

(57) ABSTRACT

Methods, probe assemblies and systems are provided for treating tissue a margins surrounding interstitial spaces created via the removal of tumors. The interstitial space may be in any tissue, e.g., breast tissue, and the interstitial space may be created by removing abnormal tissue. A hydrophilic electrode is compressed and introduced (e.g., percutaneously) into the interstitial cavity. An electrically conductive liquid (e.g., saline) is applied to the electrode, such that the electrode absorbs the electrically conductive liquid. The electrode is expanded into contact with the tissue margin, and electrical energy (e.g., radio frequency (RF) energy) is conveyed to the electrode, thereby ablating the tissue margin.

### FIELD OF THE INVENTION

The field of the invention relates generally to the structure and use of radio frequency (RF) electrosurgical devices for the treatment of tissue, and in particular, to the RF ablative treatment of tissue margins surrounding excised interstitial spaces.

### BACKGROUND

Tumors and other abnormal tissues can be treated in any one of a variety of manners. In one method, a tumor can be removed from the afflicted patient by retrieving the tumor from the surrounding tissue. For example, breast cancer, if not in an advanced stage that would otherwise require a radical mastectomy (i.e., complete removal of the breast), can be treated using a breast conserving surgical procedure, such as lumpectomy, tumorectomy, segmental mastectomy, or local excision, which involves removal of the suspect tissue and a margin of healthy tissue surrounding the suspect tissue through an open or keyhole incision. In some cases, breast tumors may be removed during a biopsy procedure, e.g., using a tissue retrieval device, such as that described in U.S. Pat. No. 6,471,659.

In any case, the excised interstitial space, which is left behind after removal of the tissue, is typically treated under the theory that a thin finite layer of cells contained within the tissue margin surrounding the interstitial space may be diseased, yet undetectable under the current range of technology, and that even a single malignant cell left in the margins of an excised interstitial space can multiply into a new tumor. Treatment of the margins of the interstitial space is key in reducing the recurrence rate of the disease.

Conventional techniques involving the post-operative treatment of the interstitial space include radiation, chemotherapy, and brachytherapy. Although general ionic radiation treatment utilizes equipment that is commonly available, it must be administered as multiple treatments over a period of weeks, and sometimes months. As a result, general radiation treatment is logistically challenging, time consuming, and costly. In addition, healthy tissue outside of the targeted zone is typically damaged during the radiation process. Focused external beam radiation therapy can be administered to minimize adverse affects to the surrounding healthy tissue. However, external beam radiation therapy utilizes less common equipment, which is typically costly, difficult to find, and/or filled to capacity.

Chemotherapy involves treating the interstitial space with toxic chemotherapeutic agents to destroy any remaining malignant cells. Due to the extreme toxicity of chemotherapeutic agents and variability in the size of the margin, however, chemotherapeutic treatment of an excised interstitial space will lead to the destruction of many healthy, and sometimes critical, cells. Also, due to the large size of the interstitial space relative to areas requiring treatment, it is difficult to obtain predictive infusion of a drug. Furthermore, filling an excised interstitial space results in the use of an excess quantity of the chemotherapeutic agent, which increases the cost of treatment. Increasing the dose of chemotherapeutic agent also increases the amount of the agent absorbed into a patient's system, making it difficult to achieve a therapeutic concentration of a drug locally at a target site within the excised interstitial space without producing unwanted systemic side effects.

Standard brachytherapy techniques require simultaneous placement of numerous catheters in the interstitial space and surrounding tissue. Placement of these catheters can be costly, cumbersome, and time-consuming. New brachytherapy methods, such as the Mammosite® Radiation Therapy System (RTS), use a balloon to deliver a conformal dose of radiation to the tissue over a treatment span of five days. To uniformly radiate the tissue margin around the interstitial space, however, it must be ensured that the balloon contacts the entirety of the wall surrounding the interstitial space. Also, even though the new brachytherapy methods focus therapy in the targeted regions, the use of radiation still poses a danger and is relatively expensive.

For this reason, it would be desirable to provide improved methods and systems for treating interstitial spaces after abnormal tissue, such as a tumor, is excised from a patient.

### SUMMARY OF THE INVENTION

In accordance with a first aspect of the present inventions, a method of treating a margin of tissue surrounding an interstitial space is provided. The interstitial space may be contained within any tissue, e.g., breast tissue, and the interstitial space may be created by removing abnormal tissue, e.g., a tumor. The method comprises introducing a compresses hydrophilic electrode within the interstitial space, e.g., by percutaneously introducing the electrode into the interstitial space. The method further comprises exposing the electrode to an electrically conductive liquid (e.g., saline), such that the electrode absorbs the electrically conductive liquid, expanding the electrode into contact with the tissue margin, and conveying electrical energy, e.g., radio frequency (RF) energy, to the expanded electrode, thereby ablating the tissue margin. Thus, although the present inventions should not be so limited, an efficient and relatively inexpensive means for treating the tissue margin surrounding an interstitial cavity is provided.

In one method, the electrode is composed of an electrically insulative material, in which case, the electrically conductive liquid solely provides an electrical path through the electrode. The electrode may be composed of any compressible/expandable material that has the capability of absorbing a sufficient amount of liquid (e.g., an amount equal to at least the weight of the electrode), such that the electrode is sufficiently electrically conductive enough to act as an ablation electrode when electrical energy is applied to it. Preferably, the electrode is expanded, such that it substantially fills the interstitial cavity. In one method, the electrode is expanded by releasing a compressive force from the electrode. The expanded electrode can have any shape, e.g., spherical, but preferably assumes a shape that allows the expanded electrode to easily conform to the tissue margin. In an optional method, a chemotherapeutic agent is conveyed from the expanded electrode to the tissue margin, thereby providing an additional means of treating the tissue margin.

In accordance with a second aspect of the present inventions, a probe assembly is provided. The probe assembly comprises a probe, which in one embodiment, is configured for being percutaneously introduced through tissue. The probe assembly further comprises a compressible/expandable tissue ablation electrode carried by the distal end of the probe, and an electrical connector carried by the proximal end of the probe. In one embodiment, the probe comprises a cannula having a lumen and an inner probe shaft disposed within the cannula lumen. In this case, the electrode is mounted on the inner probe shaft, such that the electrode can be alternately retracted within the cannula lumen and deployed from the cannula lumen.

The ablation electrode is configured for absorbing fluid, and the connector is in electrical communication with the electrode. The electrode may be composed of any compressible/expandable material that has the capability absorbing a sufficient amount of liquid (e.g., an amount equal to at least the weight of the electrode), such that the electrode is sufficiently electrically conductive enough to act as an ablation electrode when electrical energy is applied to it. In one embodiment, the electrode is composed of an electrically insulative material, in which case, the fluid, if electrically conductive, solely provides an electrical path through the electrode. The expanded electrode can have any shape, e.g., spherical, but preferably assumes a shape that allows the expanded electrode to easily conform to an interstitial cavity in which it is intended to treat. In one embodiment, the electrode is self-expanding, such that the electrode instantaneously expands upon the release of a compressive force. In an optional embodiment, the electrode is impregnated with a chemotherapeutic agent.

In accordance with a third aspect of the present inventions, a tissue ablation system is provided. The system comprises a compressible/expandable tissue ablation electrode configured for absorbing fluid, and an electrical energy source, e.g., an RF source, in electrical communication with the electrode. The ablation electrode may or may not be mounted to a probe. The details of the tissue ablation electrode can be similar to those described above. The system may optionally comprise an electrically conductive fluid source in fluid communication with the electrode.

Other and further aspects and features of the invention will be evident from reading the following detailed description of the preferred embodiments, which are intended to illustrate, not limit, the present inventions.

### BRIEF DESCRIPTION OF DRAWINGS

The drawings illustrate the design and utility of embodiment(s) of the invention, in which similar elements are referred to by common reference numerals. In order to better appreciate the advantages and objects of the invention, reference should be made to the accompanying drawings that illustrate the preferred embodiment(s). The drawings, however, depict the embodiment(s) of the invention, and should not be taken as limiting its scope. With this caveat, the embodiment(s) of the invention will be described and explained with additional specificity and detail through the use of the accompanying drawings in which:

FIG. 1 is a plan view of a tissue ablation system constructed in accordance with a preferred embodiment of the present invention;

FIG. 2 is a perspective view of a tissue ablation assembly used in the tissue ablation system of FIG. 1, particularly illustrating a retracted ablation electrode;

FIG. 3 is a perspective view of the tissue ablation assembly of FIG. 2, particularly illustrating a deployed ablation electrode;

FIG. 4 is a magnified view of the distal end of the tissue ablation assembly illustrated in FIG. 2;

FIG. 5 is a magnified view of the distal end of the tissue ablation assembly illustrated in FIG. 3;

FIG. 6 is a cross-sectional view of the ablation electrode illustrated in FIG. 3;

FIGS. 7A-7G are side views illustrating a method of ablating tissue using the tissue ablation system of FIG. 1.

### DETAILED DESCRIPTION OF THE ILLUSTRATED EMBODIMENTS

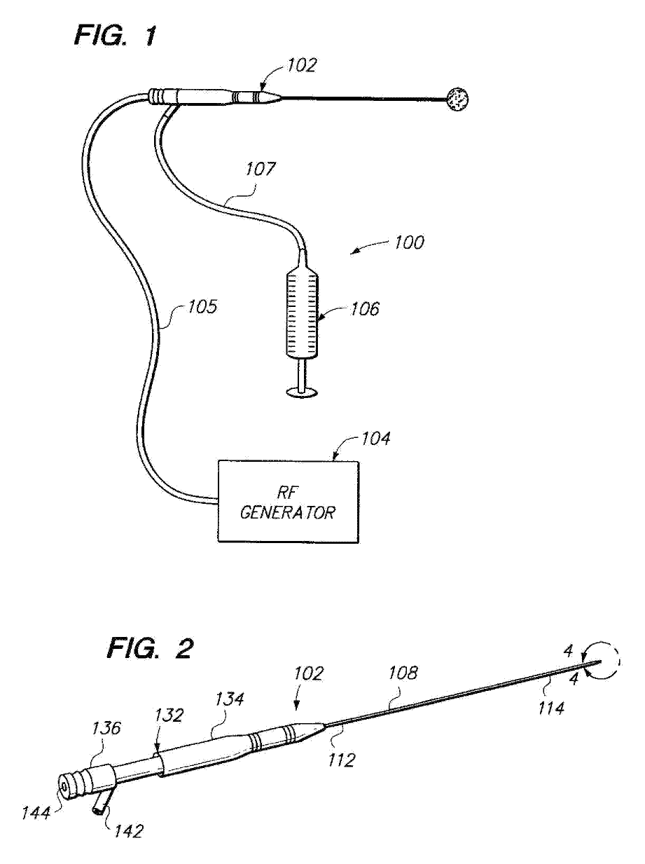

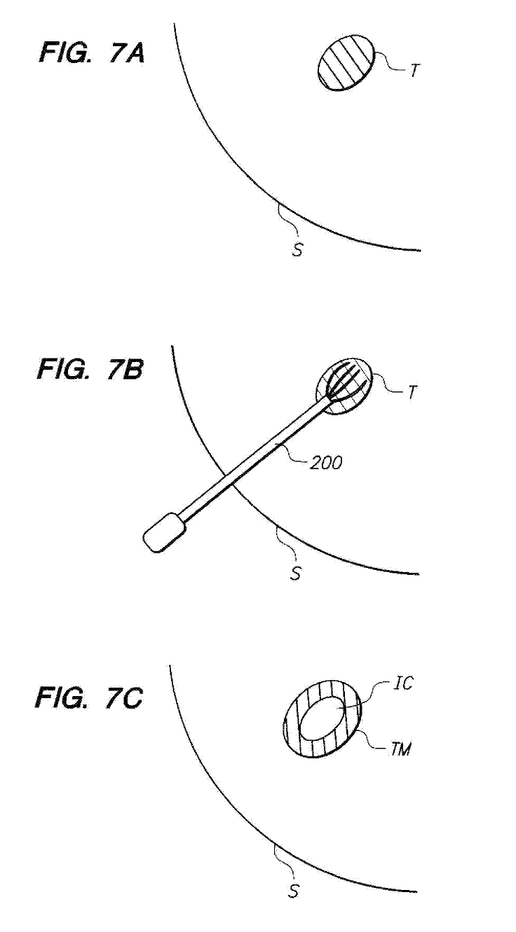

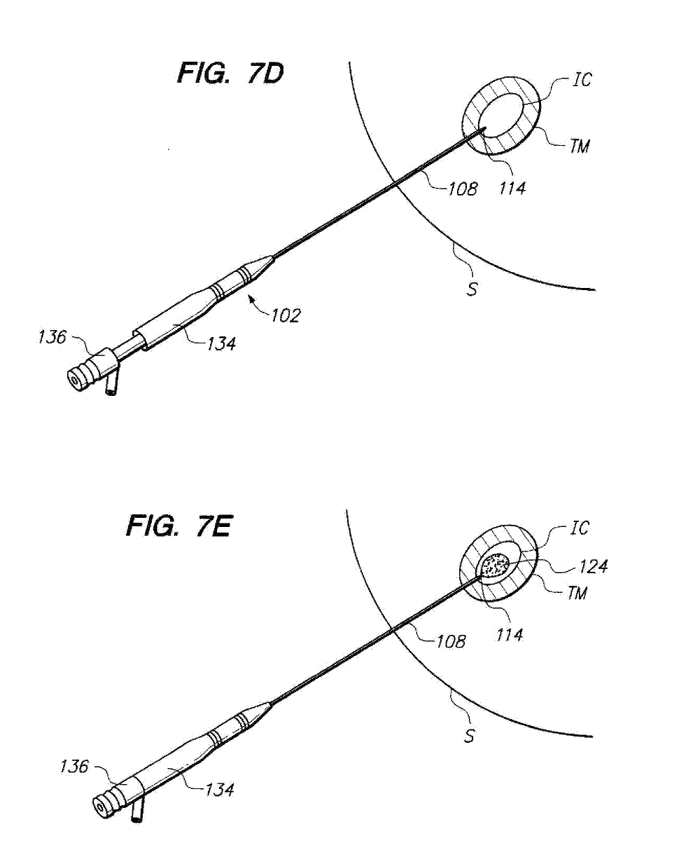

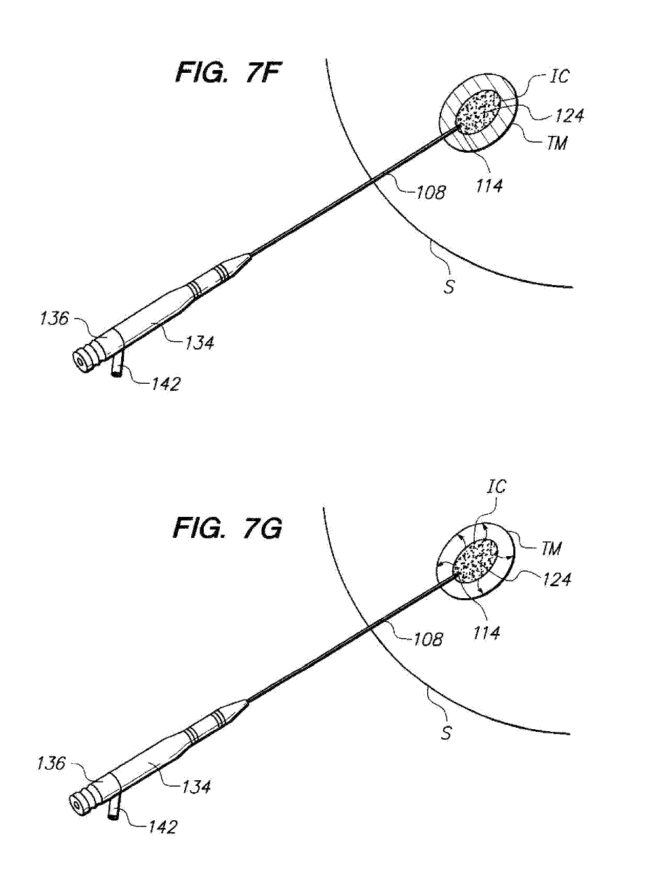

FIG. 1 illustrates a tissue ablation system 100 constructed in accordance with a preferred embodiment of the present inventions. The tissue ablation system 100 generally comprises a probe assembly 102 configured for introduction into the body of a patient for ablative treatment of a targeted margin of tissue surrounding an interstitial cavity (illustrated in FIGS. 7A-7G), an ablation energy source, and in particular a radio frequency (RF) generator 104, configured for supplying RF energy to the probe assembly 102 in a controlled manner, and an electrically conductive fluid source, and in particular a syringe 106 configured for supplying saline to the probe assembly 102 to provide an electrically conductive path for the RF energy from the probe assembly 102 to the targeted tissue margin.

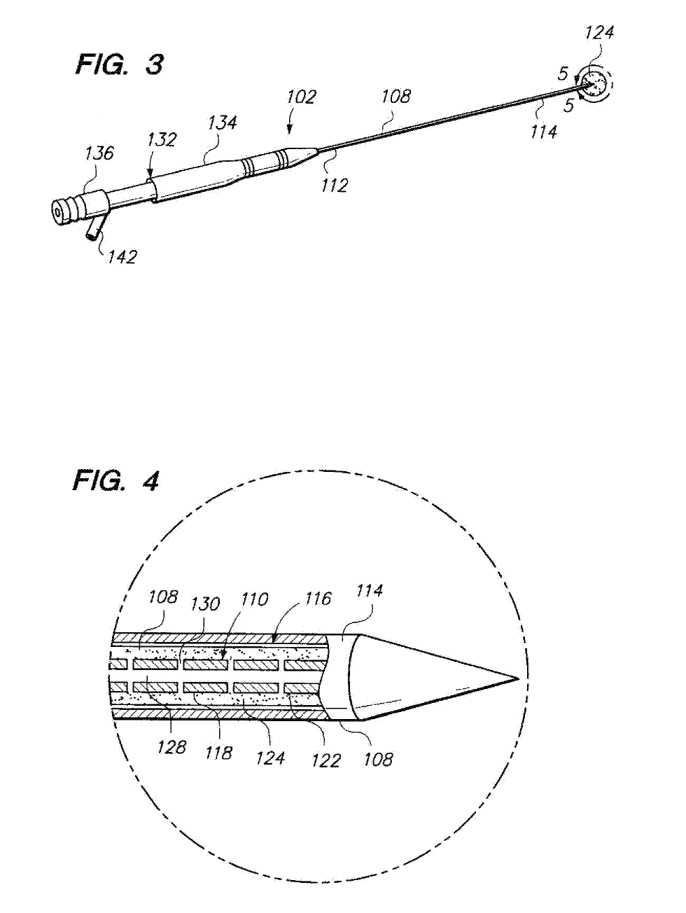

Referring specifically now to FIGS. 2 and 3, the probe assembly 102 generally comprises an elongated cannula 108 and an inner probe 110 (shown in FIG. 4) slidably disposed within the cannula 108. As will be described in further detail below, the cannula 108 serves to deliver the active portion of the inner probe 110 to the target tissue. The cannula 108 has a proximal end 112, a distal end 114, and a central lumen 116 extending through the cannula 108 between the proximal end 112 and the distal end 114. As will be described in further detail below, the cannula 108 may be rigid, semi-rigid, or flexible depending upon the designed means for introducing the cannula 108 to the target tissue. The cannula 108 has a sharpened distal tip to facilitate percutaneous introduction directly through the patient's skin. The cannula 108 is composed of a suitable material, such as plastic, metal or the like, and has a suitable length, typically in the range from 5 cm to 30 cm, preferably from 10 cm to 20 cm. If composed of an electrically conductive material, the cannula 108 is preferably covered with an insulative material (not shown). The cannula 108 has an outside diameter consistent with its intended use, typically being from 1 mm to 5 mm, usually from 1.3 mm to 4 mm. The cannula 108 has an inner diameter in the range from 0.7 mm to 4 mm, preferably from 1 mm to 3.5 mm.

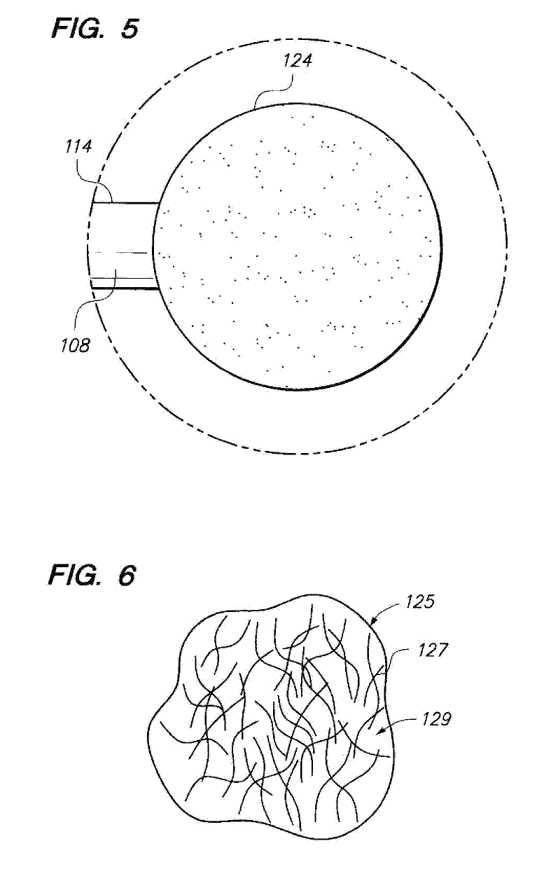

Referring further to FIG. 4, the inner probe 110 comprises a reciprocating shaft 118 having a proximal end (not shown) and a distal end 122, a tissue ablation electrode 124 mounted to the distal end 114 of the shaft 118, and a fluid delivery lumen 128 extending through shaft 118 in fluid communication the electrode 124 via lateral ports 130 extending through the wall of the shaft 118. Like the cannula 108, the shaft 118 is composed of a suitable material, such as plastic, metal or the like. It can be appreciated that longitudinal translation of the shaft 118 relative to the cannula 108 in a proximal direction retracts the electrode 126 into the distal end 114 of the cannula 108 (FIGS. 2 and 4), while longitudinal translation of the shaft 118 relative to the cannula 108 in a distal direction deploys the electrode 126 from the distal end 114 of the cannula 108 (FIGS. 3 and 5).

The electrode 124 is composed of biocompatible compressible/expandable material that allows the electrode 124 to be alternately compressed (as illustrated in FIGS. 2 and 4), so that it can be housed within the relatively small profile cannula lumen 116, and expanded (as illustrated in FIGS. 3 and 5), so that it can substantially fill the interstitial cavity when deployed from the cannula lumen 116. In the illustrated embodiment, the electrode 124 is self-expanding in that the electrode 124 automatically expands from its compressed state immediately upon the release of a compressive force.

To this end, as illustrated in FIG. 6, the compressible/expandable material comprises a skeletal structure 125 formed of elements 127 that collapse within spaces 129 between the elements 127 upon the application of a compressive force and expand upon the release of the compressive force. Preferably, the elements 127 are as thin as possible to maximize the expandability/compressibility ratio of the electrode 124, i.e., the volume of the electrode 124 in its expanded form divided by the volume of the electrode 124 in its compressed form. In this manner, the size of the cannula lumen 116, and thus the cannula 108, can be minimized as much as possible, while ensuring that the electrode 124 fills the interstitial cavity when expanded. In a preferred embodiment, the electrode 124 preferably has an expandability/compressibility ratio of more than one, preferably more than two, and most preferably more than five. The electrode 124 may also be composed of a material that expands the electrode 124 in additional volume in response absorption of the fluid. In this manner, the effective expandability/compressibility ratio of the electrode 124 is further increased.

The electrode 124 may be sized and shaped in accordance with the interstitial cavity. In the illustrated embodiment, the expanded electrode 124 is spherically-shaped. Other shapes, such as ellipsoidal, can be used, depending on the shape of the interstitial cavity. However, since the electrode 124 is preferably composed of a material that has a relatively high compliancy (i.e., it is highly compressible), any one electrode will naturally assume the shape of any variety of differently shaped interstitial cavities when expanded. Suitable expanded sizes may fall in the range of 0.5-8.0 cm, and preferably within the range of 2.0-5 cm.

The electrode 124 is hydrophilic in that it is capable of absorbing a substantial amount of fluid. It is preferred that the material used in the electrode 124 be capable of absorbing an amount of liquid at least equal to its weight, preferably an amount at least equal to at least two times its weight, and more preferably an amount at least equal to at least four times its weight. In general, the more liquid absorbed per unit weight of the electrode 124, the more electrically conductive the electrode 124 will be. To this end, the ratio between the volume of the spaces 129 and the volume of the elements 127 is maximized.

Suitable materials that can be used to construct the electrode 124 include open-cell foam (such as polyethylene foam, polyurethane foam, polyvinylchloride foam) and medical-grade sponges. In the illustrated embodiment, a foam composed of Hypol 3000 base polymer marketed by W.R. Grace & Co, an L-62 Surfactant marketed by BASF Corporation, and water is used. It has been found that the open-cell polyurethane foam marketed by Avitar, Inc. as Hydrosorb™ is especially suitable, and has been found to have an expandability/compressibility ratio of 10:1, and be capable of absorbing an amount of liquid twenty times its weight. In addition, it has been found that the use of Hydrosorb™ allows the electrode 124 to expand to 125-130% of its original uncompressed size, thereby facilitating conformance of the electrode 124 within the interstitial cavity, and thus, uniform firm contact between the electrode 124 and the tissue margin. Polyvinyl acetal sponges, such as Merocel™, marketed by Medtronic, Inc., and cellulose sponges, such as Weckcel™ are also suitable. It should be appreciated that material, other than foam or sponges may be used for the electrode 124 as long as it is capable of absorbing a sufficient amount of liquid and expands to a size necessary to fill the interstitial cavity to be treated. For example, spun-laced polyester, cotton, gauze, cellulose fiber, or the like can be used. It can be appreciated that although suitable materials used in the electrode 124 will typically be electrically insulative, the electrode 124 will become electrically conductive upon absorption of electrically conductive fluid.

For the purpose of delaying absorption of bodily fluids, the electrode 124 may optionally have a bioabsorption coating (not shown) applied to its outer surface, which controls the rate and amount of fluid that enters into the absorbent material of the electrode 124. That is, the bioabsorption coating gradually dissolves upon exposure to bodily fluid at a known rate. In this manner, the electrode 124 will not fully expand until it is desired, i.e., when the electrically conductive fluid is perfused into the electrode 124. In another optional embodiment, the electrode 124 may be impregnated with a chemotherapeutic agent (not shown). In this manner, the tissue margin, in addition to being therapeutically ablated, will be treated with the chemotherapeutic agent.

Referring back to FIGS. 2 and 3, the probe assembly 102 further comprises a handle assembly 132 composed of any suitable rigid material, such as, e.g., metal, plastic, or the like. The handle assembly 132 includes a handle sleeve 134 mounted to the proximal end 112 of the cannula 108, and a handle member 136 slidably engaged with the sleeve 134 and mounted to the proximal end (not shown) of the shaft 118. The handle member 136 further carries a perfusion port 142, which is in fluid communication with the fluid delivery lumen 128, which is further in fluid communication with the electrode 124 via the lateral ports 130. The handle member 136 carries an electrical connector 144 that is electrically coupled to the electrode 124 via the shaft 118 of the inner probe 110. In this case, the core of the shaft 118 is composed of an electrically conductive material, such as stainless steel, and the exterior of the shaft 118 is coated with an electrically insulative material. Alternatively, the electrical connector 144 may be electrically coupled to the electrode 124 via wires (not shown) extending through the shaft 118 and terminating within the electrode 124 or in the shaft distal end 122 (which will be electrically conductive in this case) on which the electrode 124 is directly mounted.

Referring back to FIG. 1, the RF generator 104 is electrically connected to the electrical connector 144 on the probe assembly 102 via a cable 105. The RF generator 104 may be a conventional RF power supply that operates at a frequency in the range from 200 KHz to 5 MHz, with a conventional sinusoidal or non-sinusoidal wave form. Such power supplies are available from many commercial suppliers, such as Valleylab, Aspen, and Bovie. Most general purpose electrosurgical power supplies, however, operate at higher voltages and powers than would normally be necessary or suitable for vessel occlusion. Thus, such power supplies would usually be operated at the lower ends of their voltage and power capabilities. More suitable power supplies will be capable of supplying an ablation current at a relatively low voltage, typically below 150V (peak-to-peak), usually being from 50V to 100V. The power will usually be from 20 W to 200 W, usually having a sine wave form, although other wave forms would also be acceptable. Power supplies capable of operating within these ranges are available from commercial vendors, such as Boston Scientific Corporation of San Jose, Calif., who markets these power supplies under the trademarks RF2000 (100 W) and RF3000 (200 W).

In the illustrated embodiment, RF current is delivered from the RF generator 104 to the electrode 124 in a monopolar fashion, which means that current will pass from the electrode 124, which is configured to concentrate the energy flux in order to have an injurious effect on the surrounding tissue, and a dispersive electrode (not shown), which is located remotely from the electrode 124 and has a sufficiently large area (typically 130 cm² for an adult), so that the current density is low and non-injurious to surrounding tissue. In the illustrated embodiment, the dispersive electrode may be attached externally to the patient, e.g., using a contact pad placed on the patient's flank.

The syringe 106 is connected to the perfusion port 142 on the probe assembly 102 via tubing 107. As briefly discussed above, the syringe 106 contains an electrically conductive fluid, such as saline. The syringe 106 is conventional and is of a suitable size, e.g., 200 ml. In the illustrated embodiment, the electrically conductive fluid is 0.9% saline. Thus, it can be appreciated the syringe 106 can be operated to convey the saline through the tubing 107, into the perfusion port 142, through the fluid delivery lumen 128 extending through the inner probe shaft 118, and into contact with the electrode 124 via the lateral ports 130. The normally electrically insulative material of the electrode 124, in turn, absorbs the saline, thereby creating an electrical path through the insulative material and transforming the electrode 124 into an electrically conductive element.

In an alternative embodiment, the probe assembly 104 and a tissue removal device are combined into a single assembly in a manner similar to co-access biopsy/ablation assemblies described in U.S. patent application Ser. No. 10/828,032, entitled “Co-Access Bipolar Ablation Probe,” and U.S. patent application Ser. No.11/030,229, entitled “Co-Access Bipolar Ablation Probe,” which are incorporated herein by reference. In these assemblies, the cannula used to introduce the inner ablation probe and the cannula used to introduce an inner tissue removal device are one in the same. That is, a single cannula is used to provide access to a treatment region for an inner tissue removal device, in addition to the inner ablation probe. In these arrangements, the cannula can be used to interchangeably introduce the inner tissue removal device and inner probe, so that a treatment region within the patient need only be accessed once. That is, an access cannula need only be percutaneously advanced through intervening tissue to the treatment region one time, since it will be used to provide access to both the tissue removal device and the inner probe.

Having described the structure of the tissue ablation system 100, its operation, along with a conventional percutaneous tissue removal device 200 (such as the En-bloc tumor removal assembly marketed by Neothermia or the MiniTome Potential marketed by Artemis), in treating targeted tissue will now be described. Although the tissue ablation system 100 and associated tissue removal device 200 lend themselves well to the treatment of tumors within breast tissue, the tissue ablation system 100 and associated tissue removal device 200 may be used to treat targeted tissue located anywhere in the body where hyperthermic exposure may be beneficial, e.g., within an organ of the body, such as the liver, kidney, pancreas, prostrate (not accessed via the urethra), and the like. The volume to be treated will depend on the size of the tumor or other lesion, typically having a total volume from 1 cm³ to 150 cm³, and often from 2 cm³ to 35 cm³. The peripheral dimensions of the treatment region may be regular, e.g., spherical or ellipsoidal, but will more usually be irregular. The treatment region may be identified using conventional imaging techniques capable of elucidating a target tissue, e.g., tumor tissue, such as ultrasonic scanning, magnetic resonance imaging (MRI), computer-assisted tomography (CAT), fluoroscopy, nuclear scanning (using radiolabeled tumor-specific probes), and the like. Preferred is the use of high resolution ultrasound of the tumor or other lesion being treated, either intraoperatively or externally.

Referring now to FIGS. 7A-7G, the operation of the tissue removal device and tissue ablation system 100 is described in treating a tumor T located beneath the skin or an organ surface S of a patient (FIG. 7A). First, the tissue removal device 200 is percutaneously introduced within the tumor T (FIG. 7B), and then operated in a conventional manner to remove the tumor T from the patient, thereby creating an interstitial cavity IC surrounding by a tissue margin TM (FIG. 7C).

Next, the probe assembly 102, while the electrode 124 is retracted and placed in its compressed state within the cannula 108, is introduced within the tissue, so that the distal end 114 of the cannula 108 is located within the interstitial cavity IC (FIG. 7D). Introduction of the probe assembly 102 can be performed transoperatively, i.e., soon after the tumor T is removed, or post-operatively, i.e., a significant amount of time (perhaps days) after the tumor T has been removed. In the latter case, a drainage tube is typically placed within the tissue to drain any fluid from the interstitial cavity IC, thereby maintaining a convenient path through which the probe assembly 104 can be later introduced.

This can be accomplished using any one of a variety of techniques. In some cases, the probe assembly 102 may be introduced to the target site TS percutaneously directly through the patient's skin or through an open surgical incision. In this case, the cannula 108 may have a sharpened tip, e.g., in the form of a needle, to facilitate introduction to the treatment region TR. In such cases, it is desirable that the cannula 108 be sufficiently rigid, i.e., have a sufficient column strength, so that it can be accurately advanced through tissue. Of course, if the cannula 108 is introduced through the same path initially created by the tissue removal device 200, access through the patient's skin will have already been provided.

Alternatively, the cannula 108 may be introduced using an internal stylet that is subsequently exchanged for the inner probe 110. In this latter case, the cannula 108 can be relatively flexible, since the initial column strength will be provided by the stylet. More alternatively, a component or element may be provided for introducing the cannula 108 to the interstitial cavity IC. For example, a conventional sheath and sharpened obturator (stylet) assembly can be used to access the interstitial cavity IC. The assembly can be positioned under ultrasonic or other conventional imaging, with the obturator/stylet then removed to leave an access lumen through the sheath. The probe assembly 104 can then be introduced through the sheath lumen, so that the distal end 114 of the cannula 108 advances from the sheath into the interstitial cavity IC. In the case of the co-access assembly described above, the same cannula used to introduce the tissue removal device 200 will be used to introduce the inner probe 110, thereby obviating the need to subsequently reintroduce another cannula through the tissue.

In any event, after the cannula 108 is properly placed, the shaft 118 is distally advanced relative to the cannula 108 in a stable position to deploy the electrode 124 out from the distal end 114 of the cannula 108 (FIG. 7E). This can be accomplished either by holding the cannula 108 via the handle sleeve 134 and distally advancing the shaft 118, or by holding shaft 118 via the handle member 136 and proximally advancing the cannula 108 via the handle sleeve 134. Depending upon the architecture and composition of the electrode 124, the electrode 124 may partially or completely self-expand upon its deployment from the cannula 108, or may remain in its compressed state. In the illustrated method, the electrode 124 is shown partially expanded, so that it only partially fills the entire interstitial cavity IC.

Next, the syringe 106 and associated tubing 107 (shown in FIG. 1) are connected to the perfusion port 142 on the handle member 136, and the syringe 106 operated, such that the saline is conveyed under positive pressure, through the tubing 107, and into the perfusion port 142 on the handle member 136. The saline then travels through the lumen 128 in the inner probe shaft 118, through the lateral ports 130 (shown in FIG. 4), and into contact with the ablation electrode 124, where it is absorbed by the electrode 124. As a result, the ablation electrode 124 becomes electrically conductive, and if not completely expanded already, will expand into firm and uniform contact with the tissue margin TM (FIG. 7F). Notably, even if the shape of the expanded electrode 124 does not match the shape of the interstitial cavity IC, the pliability of the electrode 124 allows it to easily conform to the tissue margin TM.

Next, the RF generator 104 and associated cable 105 (shown in FIG. 1) are connected to the connector 144 on the handle member 136, and then operated to ablate the tissue margin TM (FIG. 7G). If the electrode 124 is impregnated with a chemotherapeutic agent, any gaseous substances created as a result of the thermal ablation process will escape from the interstitial cavity IC outward through the tissue margin TM, thereby carrying the chemotherapeutic agent with it into the tissue margin TM where chemotherapy is needed. Thus, any pathological agents not otherwise killed by the ablation process will be killed by the chemotherapy. It should also be noted that the heat created by the thermal ablation process increases the metabolic process of the tissue margin TM, thereby facilitating uptake of the chemotherapeutic agent within the tissue. The probe assembly 104 is then removed from the patient.

It should be noted that although use of a compressible/expandable hydrophilic electrode has been described in terms of percutaneous delivery systems and methods, such electrodes can be introduced through open surgical incisions if desired. For example, an open incision can be made through the skin S of the patient, so that the tumor T can be removed via conventional surgical means. The electrode can then be placed within the resulting interstitial cavity IC (either with or without the aid of a probe), and RF energy applied to the electrode to ablate the targeted tissue margin TM. If a probe is not used, insulated RF wires can be connected directly to the electrode, and an electrically conductive fluid can be directly applied to the electrode simply by directly perfusing the electrode with, e.g., a syringe. Depending upon the size of the surgical opening and the size of the fully expanded electrode, the electrode may not need to be compressed prior to its introduction within the interstitial cavity IC.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

1. A method of treating a margin of tissue surrounding an interstitial space, comprising:
introducing a compressed hydrophilic electrode within the interstitial space;
exposing the electrode to an electrically conductive liquid, such that the electrode absorbs the electrically conductive liquid;
expanding the electrode into contact with the tissue margin; and
conveying electrical energy to the expanded electrode, thereby ablating the tissue margin.

2. The method of claim 1, wherein the compressed electrode is percutaneously introduced into the interstitial space.

3. The method of claim 1, wherein the electrode is composed of an electrically insulative material, and the absorbed electrically conductive liquid provides an electrically conductive path through the electrode.

4. The method of claim 1, wherein expansion of the electrode comprises solely releasing a compressive force from the electrode.

5. The method of claim 1, wherein the expanded electrode substantially fills the interstitial space.

6. The method of claim 1, wherein the electrode absorbs an amount of the electrically conductive fluid equal to at least the weight of the electrode.

7. The method of claim 1, wherein the electrically conductive liquid comprises saline.

8. The method of claim 1, wherein the electrode expands in response to absorption of the electrically conductive liquid.

9. The method of claim 1, wherein the electrical energy is radio frequency (RF) energy.

10. The method of claim 1, further comprising removing abnormal tissue to create the interstitial space.

11. The method of claim 1, wherein the interstitial space is within breast tissue.

12. The method of claim 1, further comprising conveying a chemotherapeutic agent from the expanded electrode to the tissue margin.

13. A probe assembly, comprising:
a probe having a proximal end and a distal end;
an compressible/expandable tissue ablation electrode carried by the distal probe end, the electrode configured for absorbing fluid, whereby the electrode expands to a size substantially greater than an original uncompressed size of the electrode; and
an electrical connector carried by the proximal probe end, the connector in electrical communication with the electrode.

14. The probe assembly of claim 13, wherein the probe is configured for being percutaneously introduced through tissue.

15. The probe assembly of claim 13, wherein the probe comprises a cannula having a lumen and an inner probe shaft disposed within the cannula lumen, and wherein the electrode is mounted on the inner probe shaft, such the electrode can be alternately retracted within the cannula lumen and deployed from the cannula lumen.

16. The probe assembly of claim 13, wherein the electrode is composed of an electrically insulative material.

17. The probe assembly of claim 13, wherein the electrode is self-expanding.

18. The probe assembly of claim 13, wherein the electrode comprises a network of spaces configured to fill with the fluid.

19. The probe assembly of claim 13, wherein the electrode is configured for absorbing an amount of the fluid equal to at least the weight of the electrode.

20. The probe assembly of claim 13, further comprising an electrical energy source electrically coupled to the electrical connector.

21. The probe assembly of claim 13, further comprising a perfusion port carried by the proximal shaft end, the perfusion port in fluid communication with the electrode.

22. The probe assembly of claim 13, further comprising a chemotherapeutic agent impregnated in the electrode.

23. A tissue ablation system, comprising:
a compressible/expandable tissue ablation electrode configured for absorbing fluid, whereby the electrode expands to a size substantially greater than an original uncompressed size of the electrode; and
an electrical energy source in electrical communication with the electrode.

24. The tissue ablation system of claim 23, wherein the electrode is composed of an electrically insulative material.

25. The tissue ablation system of claim 23, wherein the electrode is self-expanding.

26. The tissue ablation system of claim 23, wherein the electrode comprises a network of spaces configured to fill with the fluid.

27. The tissue ablation system of claim 23, wherein the electrode is configured for absorbing an amount of the electrically conductive fluid equal to at least the weight of the electrode.

28. The tissue ablation system of claim 23, wherein the electrode is configured for expanding in response to absorption of the fluid.

29. The tissue ablation system of claim 23, wherein the energy source comprises a radio frequency (RF) source.

30. The tissue ablation system of claim 23, further comprising an electrically conductive fluid source in fluid communication with the electrode.

31. The tissue ablation system of claim 23, further comprising a chemotherapeutic agent impregnated in the electrode.
